(19) United States
(12) Patent Application Publication
(10) Pub. No.: US 2026/0212504 A1
(43) Pub. Date: Jul. 23, 2026

(54) METHOD FOR DETERMINING FRAME THROUGH CARDIAC CYCLE PREDICTION, METHOD FOR GENERATING 3D DATA, AND ELECTRONIC DEVICE PERFORMING THE SAME

(71) Applicant: Medipixel, Inc., Seoul (KR)

(72) Inventors: Donggyu KIM, Seoul (KR); Hyun-woo KIM, Seoul (KR); Youngrak CHOI, Seoul (KR)

(73) Assignee: Medipixel, Inc., Seoul (KR)

(21) Appl. No.: 19/451,775

(22) Filed: Jan. 16, 2026

(30) Foreign Application Priority Data
Jan. 20, 2025 (KR) 10-2025-0007743

(51) Int. Cl. G06T 7/00; G06T 7/11; G06T 7/20; G06T 7/70; G06T 17/00

(52) U.S. Cl. CPC G06T 7/0014; G06T 7/11; G06T 7/20; G06T 7/70; G06T 17/00; G06T 2207/30101

(57) ABSTRACT

Provided is an electronic device including a processor and a memory connected to the processor, wherein the memory is configured to store a program, wherein the processor is configured to execute the program, and wherein, when the program is executed, the electronic device is configured to obtain a plurality of image frames, determine one of the plurality of image frames as a reference frame, determine vessel points in the reference frame, for each of the plurality of image frames, determine coordinate data of the vessel points, and determine a key frame corresponding to a specific cardiac phase based on a time-series variation of the coordinate data.

### CROSS-REFERENCE TO RELATED APPLICATION

This application claims priority to and the benefit thereof under 35 U.S.C. § 119 to Korean Patent Application No. 10-2025-0007743 filed in the Ministry of Intellectual Property on Jan. 20, 2025, the entire contents of which are incorporated herein by reference.

### BACKGROUND

### (a) Field

The disclosure relates to a frame determination method through cardiac-cycle prediction, a 3D data generation method, and an electronic device performing the same.

### (b) Description of the Related Art

In the field of medical image processing, a technology for reconstructing a 3D vascular shape plays a key role in diagnosis of vascular diseases and establishment of treatment plans. This technology enables medical staff to precisely evaluate a patient's vascular condition by visualizing a complex vascular structure three-dimensionally. For example, accurately reconstructing a vascular structure of a moving organ such as a heart is essential for early detection of vascular diseases and effective treatment.

A 3D vascular shape reconstruction mainly uses contrast images obtained through an X-ray-based imaging technique. The contrast images are captured at various angles and are used to identify a three-dimensional shape and structure of a vessel.

A conventional 3D vascular shape reconstruction method is performed in a manner of reconstructing a 3D structure of a vessel by combining two contrast images. In this process, a specific frame is selected from each contrast image and then combined, thereby forming a three-dimensional shape of a target vessel. However, it is difficult to obtain precise 3D data due to, for example, a case in which a vessel continuously moves and deforms due to movement of tissue, a case in which distortion or noise occurs depending on an imaging environment, and the like.

### SUMMARY

Some embodiments may provide a method and an apparatus for determining a cardiac cycle from a plurality of image frames without use of additional data.

Some embodiments may provide a method and an apparatus for determining key frames corresponding to a specific cardiac phase from a plurality of image frames without use of additional data.

Some embodiments may provide a method and an apparatus for generating 3D data based on key frames.

However, the technical problems to be solved by the present invention are not limited to those described above, and may comprise other technical problems that are not mentioned but can be clearly understood by a person having ordinary skill in the art from the following description.

According to an aspect of an embodiment, an electronic device may include a processor; and a memory connected to the processor, wherein the memory is configured to store a program, wherein the processor is configured to execute the program, and wherein, when the program is executed, the electronic device is configured to: obtain a plurality of image frames; determine one of the plurality of image frames as a reference frame; determine vessel points in the reference frame; for each of the plurality of image frames, determine coordinate data of the vessel points; and determine a key frame corresponding to a specific cardiac phase based on a time-series variation of the coordinate data.

According to an aspect of an embodiment, an electronic device may include a processor; and a memory connected to the processor, wherein the memory is configured to store a program, wherein the processor is configured to execute the program, and wherein, when the program is executed, the electronic device is configured to: obtain a first image frame and a second image frame captured at different times and different positions; determine a first key frame based on point tracking of the first image frame; determine a second key frame corresponding to the first key frame based on point tracking of the second image frame; and generate 3D data based on the first key frame and the second key frame.

According to an aspect of an embodiment, a frame determination method may include: obtaining a plurality of image frames; determining one of the plurality of image frames as a reference frame; determining vessel points in the reference frame; for each of the plurality of image frames, determining coordinate data of the vessel points; and determining a key frame corresponding to a specific cardiac phase based on a time-series variation of the coordinate data.

According to an aspect of an embodiment, a 3D data generation method may include: obtaining a first image frame and a second image frame captured at different times and different positions; determining a first key frame based on point tracking of the first image frame; determining a second key frame corresponding to the first key frame based on point tracking of the second image frame; and generating 3D data based on the first key frame and the second key frame.

Additional aspects may be set forth in part in the description which follows and, in part, may be apparent from the description, and/or may be learned by practice of the presented embodiments.

### BRIEF DESCRIPTION OF THE DRAWINGS

The above and other aspects, features, and advantages of certain embodiments of the present disclosure will become apparent from the following description taken in conjunction with the accompanying drawings, in which:

FIG. 1 is a schematic block diagram of a computing system according to one embodiment.

FIG. 2 is a block diagram of an electronic device according to one embodiment.

FIG. 3 is a flowchart of a frame determination method according to one embodiment.

FIG. 4 is a diagram for describing a plurality of image frames according to one embodiment.

FIG. 5 is a diagram for describing a reference frame according to one embodiment.

FIG. 6 is a diagram for describing a centerline according to one embodiment.

FIG. 7 is a diagram for describing point tracking according to one embodiment.

FIG. 8 is a diagram for describing an X-axis coordinate change of a vessel point over time according to one embodiment.

FIG. 9 is a diagram for describing a configuration for determining a cardiac cycle from a coordinate change of a vessel point according to one embodiment.

FIG. 10 is a diagram for describing a Y-axis coordinate change of a vessel point over time according to one embodiment.

FIG. 11 is a diagram for describing a configuration for determining a cardiac cycle from a coordinate change of a vessel point according to one embodiment.

FIG. 12 is a diagram for describing a coordinate change of a vessel point over time according to one embodiment.

FIG. 13 is a diagram for describing a configuration for determining a specific cardiac phase from a coordinate change of a vessel point according to one embodiment.

FIG. 14 is a flowchart for describing a noise frame determination method according to one embodiment.

FIG. 15 is a diagram for describing a noise frame according to one embodiment.

FIG. 16 is a diagram for describing a valid frame according to one embodiment.

FIG. 17 is a graph for describing a valid frame determination method according to one embodiment.

FIG. 18 is a flowchart of a 3D data generation method according to one embodiment.

FIG. 19 is a block diagram of an imaging system according to one embodiment.

FIG. 20 is a diagram for describing a structure of an imaging system according to one embodiment.

### DETAILED DESCRIPTION

In the following detailed description, only certain embodiments of the present invention have been shown and described, simply by way of illustration. As those skilled in the art would realize, the described embodiments may be modified in various different ways, all without departing from the spirit or scope of the present invention.

The drawings and description are to be regarded as illustrative in nature and not restrictive. Like reference numerals designate like elements throughout the specification. The sequence of operations or steps is not limited to the order presented in the claims or figures unless specifically indicated otherwise. The order of operations or steps may be changed, several operations or steps may be merged, a certain operation or step may be divided, and a specific operation or step may not be performed.

As used herein, the singular forms “a” and “an” are intended to include the plural forms as well, unless the context clearly indicates otherwise. Although the terms first, second, and the like may be used herein to describe various elements, components, steps and/or operations, these terms are only used to distinguish one element, component, step or operation from another element, component, step, or operation.

As used herein, each of such phrases as “A or B,” “at least one of A and B,” “at least one of A or B,” “A, B, or C,” “at least one of A, B, and C,” and “at least one of A, B, or C,” may include any possible combination of the items enumerated together in a corresponding one of the phrases.

Reference throughout the present disclosure to “one embodiment,” “an embodiment,” “an example embodiment,” or similar language may indicate that a particular feature, structure, or characteristic described in connection with the indicated embodiment is included in at least one embodiment of the present disclosure. Thus, the phrases “in one embodiment,” “in an embodiment,” “in an example embodiment,” and similar language throughout this disclosure may, but do not necessarily, all refer to the same embodiment.

Hereinafter, various embodiments of the present disclosure are described with reference to the accompanying drawings.

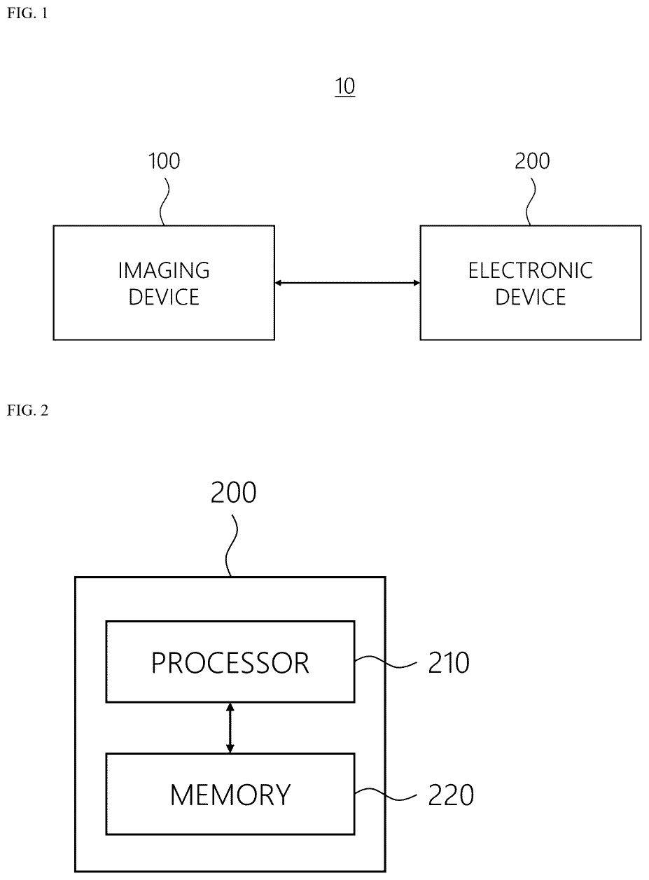

FIG. 1 is a schematic block diagram of a computing system according to one embodiment, and FIG. 2 is a block diagram of an electronic device according to one embodiment.

Referring to FIG. 1, a computing system 10 according to one embodiment may obtain a vascular image of a first user (object), may perform an operation based on the obtained vascular image, and may display a processed image. The computing system 10 may provide the processed image to a second user. For example, the first user may be a patient, and the second user may be medical staff.

In one embodiment, the computing system 10 may capture a cardiovascular system of the first user to obtain a plurality of cardiovascular images. The plurality of cardiovascular images are stereo images, and each cardiovascular image may be composed of a plurality of image frames captured in a time series. The computing system 10 may generate three-dimensional vascular data based on the plurality of cardiovascular images. However, embodiments are not necessarily limited thereto, and the computing system 10 may capture a blood vessel that is a target of angiography, such as a cerebral blood vessel, a gastrointestinal blood vessel, or the like.

A computing system 10 according to one embodiment includes an imaging device 100 and an electronic device 200. The imaging device 100 may be an imaging device configured to capture the first user to obtain an image. For example, the imaging device 100 may be X-ray imaging equipment (for example, C-arm X-ray equipment), angiographic imaging equipment (for example, angio equipment), optical coherence tomography (OCT) equipment, computed tomography (CT) equipment, or magnetic resonance imaging (MRI) equipment, magnetic resonance angiography (MRA) equipment, but embodiments are not necessarily limited thereto, and may be implemented as various devices configured to capture a blood vessel of the first user.

The imaging device 100 may capture the first user at a plurality of imaging points and may obtain a plurality of images. In one embodiment, the imaging device 100 may capture the first user while rotating around the first user. In another embodiment, the imaging device 100 may rotate the first user and may capture the rotating first user. In one embodiment, the imaging device 100 may be implemented as a mono-plane device. However, embodiments are not necessarily limited thereto, and the imaging device 100 may also be implemented as a bi-plane device.

The imaging device 100 may transmit the obtained plurality of images to the electronic device 200. Here, images obtained and transmitted by the imaging device 100 may include an X-ray image, an ultrasound (or sonography) image, a computed tomography (CT) image, a positron emission tomography (PET) image, a magnetic resonance imaging (MRI) image, a functional magnetic resonance imaging (fMRI) image, a digital pathology whole slide image (WSI), a digital breast tomosynthesis (DBT) image, and the like.

The electronic device 200 may be a computing device configured to determine a key frame corresponding to a specific cardiac phase based on an image received from the imaging device 100. The specific cardiac phase may refer to a diastolic phase or a systolic phase. In one embodiment, the received image may be a two-dimensional image, but embodiments are not necessarily limited thereto.

The electronic device 200 may determine a cardiac cycle through point tracking in the received image. The electronic device 200 may determine a key frame based on the cardiac cycle. The electronic device 200 may generate three-dimensional data based on the key frame. The electronic device 200 may generate a fractional flow reserve (FFR) value based on the three-dimensional data.

The electronic device 200 may be an Artificial Intelligence AI device, a Personal Computer PC, a laptop computer, a mobile phone, a smart phone, a tablet PC, a wearable device, a medical imaging device, a healthcare device, or the like.

In some embodiments, the computing system 10 may further include a server such as an AI server or a data center, and the electronic device 200 may be implemented to communicate with the server. That is, the electronic device 200 may use an artificial neural network of the server.

Referring to FIG. 2, an electronic device 200 according to one embodiment may include a processor 210 and a memory 220 connected to the processor 210. The memory 220 may be configured to store a program. The processor 210 may be configured to execute the program of the memory 220. When the program is executed, steps of the frame determination method according to embodiments may be implemented.

The memory 220 may be configured to store images and frames received from the imaging device 100. At least one of the images and frames stored in the memory 220 may be loaded by the processor 210 and may be used for key frame determination.

The processor 210 may perform an operation based on an image received from the imaging device 100 and may determine a key frame. The processor 210 may obtain a first image and a second image that are stereo images. That is, the first image and the second image may be images captured by the imaging device 100 with different imaging parameters. The imaging parameters may include a capturing position of the imaging device 100, a capturing angle, a capturing distance, a position and an angle of a table on which the first user is placed, a focal length, a baseline distance, a capturing direction (axial rotation information), a pixel spacing (pixel space), and relative position and direction information between capturing sources.

The processor 210 may obtain a plurality of image frames included in the first image. The processor 210 may determine a reference frame among the plurality of image frames. The processor 210 may determine vessel points in the reference frame. The processor 210 may determine the vessel points based on vascular information. In one embodiment, the processor 210 may determine a centerline or a contour in the reference frame and may determine vessel points in the centerline or the contour.

The processor 210 may track coordinate changes of vessel points in the plurality of image frames. For example, the processor 210 may determine coordinate data of vessel points determined in the reference frame in each image frame. Through the coordinate data, the processor 210 may determine how the vessel points move in each image frame. The processor 210 may determine a key frame corresponding to a specific cardiac phase based on time-series variation of the coordinate data. In a similar manner, the processor 210 may also determine a key frame for the second image.

The processor 210 may generate three-dimensional data using key frames of the first image and the second image. The processor 210 may generate or calculate an FFR value based on the three-dimensional data. In this manner, the electronic device 200 may omit a process in which the second user manually selects a frame corresponding to a specific cardiac phase by extracting a key frame through point tracking in images. Further, since the electronic device 200 may extract a key frame using only information of an image without additional data (for example, an electrocardiogram ECG, etc.), the electronic device 200 may generate three-dimensional data rapidly and precisely even with reduced data and computational resources.

Although FIG. 2 illustrates that the electronic device 200 includes the processor 210 and the memory 220 for convenience of description, embodiments are not necessarily limited thereto, and the electronic device 200 may be implemented to further include at least one other component. For example, the electronic device 200 may further include components such as a user interface, a communication module, and a display. The user interface may be a component for an interface between an input/output device and the electronic device 200. The communication module may be a component for communication with an external device of the electronic device 200 (for example, the imaging device 100, a server, etc.). The display may be a component for displaying an output of the processor 210 (for example, a reference frame, a key frame, three-dimensional data, an FFR value, etc.).

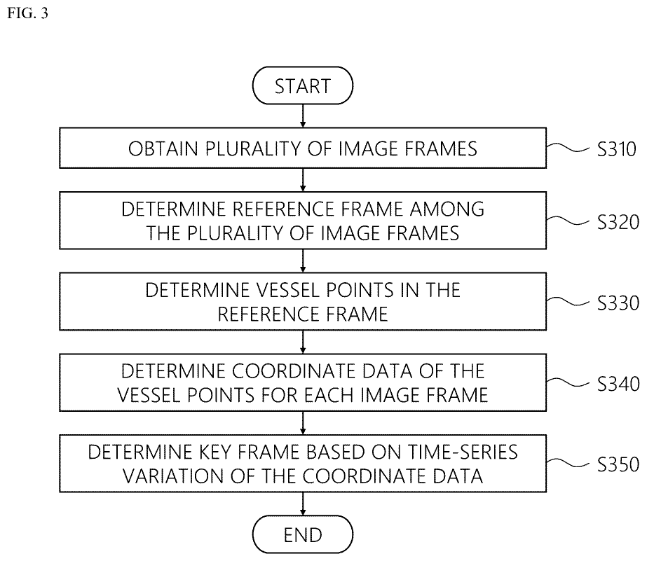

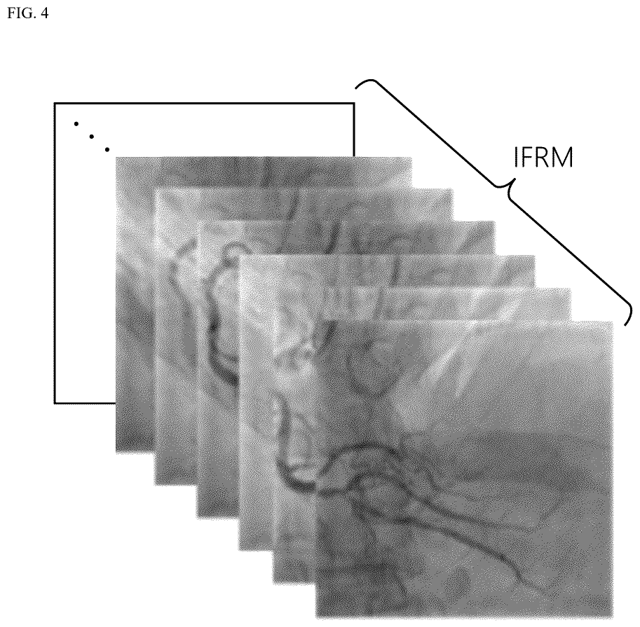

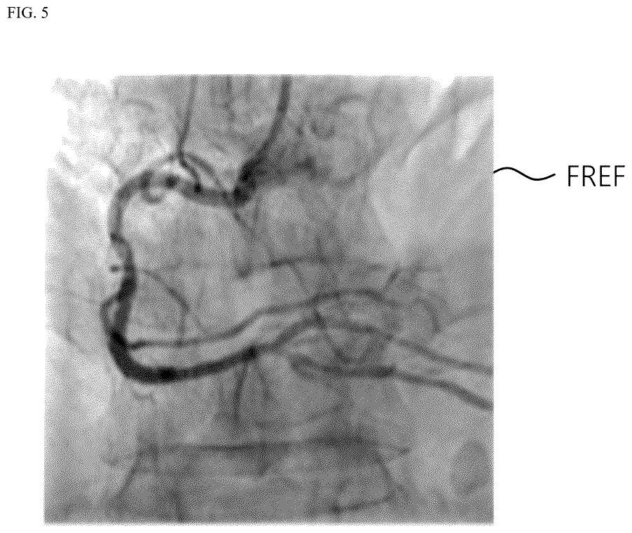

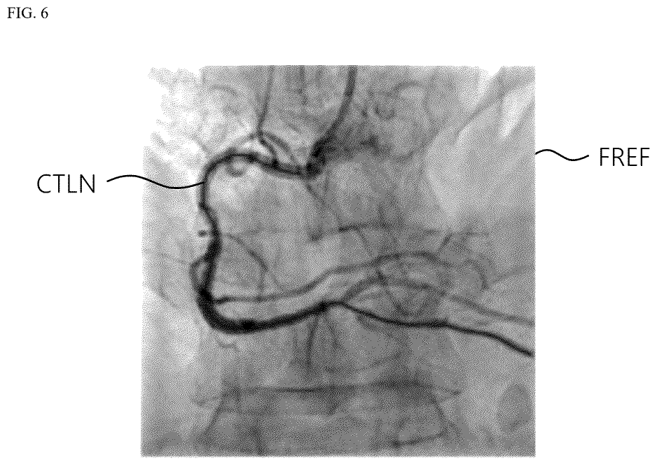

FIG. 3 is a flowchart of a frame determination method according to one embodiment, FIG. 4 is a diagram for describing a plurality of image frames according to one embodiment, FIG. 5 is a diagram for describing a reference frame according to one embodiment, and FIG. 6 is a diagram for describing a centerline according to one embodiment.

Referring to FIG. 3, a frame determination method according to one embodiment may be performed by an electronic device (for example, 200 of FIGS. 1 and 2). The electronic device may obtain a plurality of image frames (S310). The electronic device may obtain a stereo image from an imaging device. The stereo image may include a first image and a second image captured at different positions and different angles. Each of the first image and the second image may include a plurality of image frames captured in a time series. The plurality of image frames may be as illustrated in FIG. 4. Referring to FIG. 4, the electronic device may receive a plurality of image frames IFRM from an imaging device (for example, 100 of FIGS. 1 and 2).

The electronic device may determine one of the plurality of image frames as a reference frame (S320). The plurality of image frames may be captured at different times so that at least one of a contrast agent distribution or a blood vessel position may be different. The electronic device may determine a contrast agent distribution level of the plurality of image frames. For example, the electronic device may quantitatively evaluate the contrast agent distribution level by calculating a distribution pattern of a contrast agent in each image frame. The electronic device may generate a brightness histogram in units of pixels (or based on pixel intensities) from an image frame and may determine the contrast agent distribution level based on a ratio of pixels having a predetermined brightness value or more. According to an embodiment, the electronic device may also use a first artificial neural network model trained to output the contrast agent distribution level from an image frame. The first artificial neural network model may be pre-trained using training data including an image frame and the contrast agent distribution level.

The electronic device may determine a vessel position score of the plurality of image frames. The electronic device may identify a blood vessel in each image frame. For example, the electronic device may use an algorithm such as edge detection or contour extraction. The electronic device may determine the vessel position score based on at least one of an alignment degree, continuity, curvature, and density of the identified blood vessel. The electronic device may determine a frame having the highest vessel position score as the reference frame.

The alignment degree may indicate how far a target vessel is from a center (focus area) of an image frame. For example, the electronic device may determine vessel points from a blood vessel and may determine a distance of the vessel points from the center of the image frame. As the distance value is smaller, the alignment degree may be higher, and as the distance value is larger, the alignment degree may be lower.

The continuity may indicate a degree to which a blood vessel is connected without being interrupted. For example, the electronic device may determine a total length of a blood vessel in an image frame and may determine continuity based on the total length. As the total length is longer, continuity may be higher, and as the total length is shorter, continuity may be lower.

The curvature may be a degree to which a blood vessel is bent, and may indicate a degree of distortion. The electronic device may determine curvatures of adjacent points among vessel points in an image frame. The electronic device may determine the degree of distortion based on a ratio of points having a curvature equal to or greater than a predetermined curvature in the image frame. In an image frame in which a ratio of points having a large curvature is high, the degree of distortion may be severe, and in an image frame in which a ratio of points having a large curvature is low, the degree of distortion may be low.

The density may indicate how densely a blood vessel is concentrated in a specific region. The electronic device may divide an image frame into a plurality of cells and then may determine density by calculating a number of blood vessel pixels in each cell. The electronic device may calculate density of an individual cell by dividing the number of blood vessel pixels of one cell by a total number of pixels of the cell. The electronic device may calculate a density score of an image frame based on densities of all cells. For example, when density is high and uniform in a specific region among the plurality of cells of the image frame, the electronic device may assign a higher density score to the image frame. When there are many cells having density higher than a specific reference (or reference value) so that distribution is unbalanced, the electronic device may set the density score of the image frame to be low.

The electronic device may determine the reference frame based on at least one of the contrast agent distribution level and the vessel position score in the plurality of image frames. In one embodiment, the electronic device may use an artificial neural network to determine the reference frame from the plurality of image frames. The artificial neural network may be pre-trained using training data composed of the plurality of image frames and the reference frame.

The reference frame determined by the electronic device according to one embodiment may be as illustrated in FIG. 5. Referring to FIG. 5, the electronic device according to one embodiment may identify a blood vessel in each of the plurality of image frames IFRM of FIG. 4 and may determine the vessel position score based on at least one of an alignment degree, continuity, curvature, and density of the identified blood vessel. The electronic device may determine a frame having the highest vessel position score as a reference frame FREF.

The electronic device may determine vessel points in the reference frame (S330). The electronic device may perform segmentation of a blood vessel region in the reference frame. For example, the electronic device may identify and separate the blood vessel region (or a blood vessel mask) by analyzing features such as an intensity, color, brightness, or texture of pixels constituting the reference frame. The electronic device may determine a skeleton in the segmented blood vessel region. For example, the electronic device may determine the skeleton by gradually reducing an outline of the blood vessel region through a thinning algorithm. The electronic device may determine vessel points based on the skeleton.

The electronic device according to one embodiment may determine vessel points based on vascular information. For example, the electronic device may determine vessel points from a centerline or a contour. The electronic device may determine a centerline in the reference frame by using a VMTK (Vascular Modeling Toolkit) library. However, embodiments are not necessarily limited thereto, and the centerline may also be determined by using other vision libraries, image operations (such as an Otsu algorithm), graph modeling, deep learning, physical models, or the like.

A centerline determined in the reference frame by the electronic device according to one embodiment may be as illustrated in FIG. 6. Referring to FIG. 6, the electronic device may determine a centerline CTLN of a target vessel in the reference frame FREF. The electronic device may determine vessel points based on the centerline CTLN.

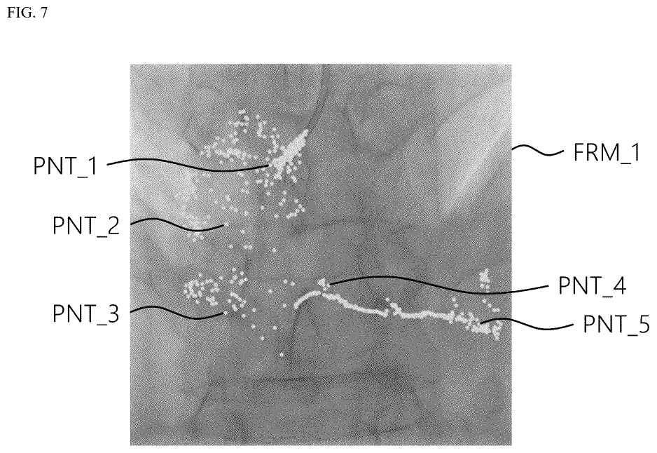

FIG. 7 is a diagram for describing point tracking according to one embodiment.

Referring to FIG. 3, an electronic device may determine coordinate data of vessel points for each of a plurality of image frames (S340). The electronic device may extract vessel points based on vascular information and may track the vessel points in the plurality of image frames captured in time series. The electronic device may determine the coordinate data according to changes of the vessel points.

The electronic device may determine the coordinate data by tracking a positional change of vessel points in the plurality of image frames by using a motion tracking model. For example, the motion tracking model may be implemented as an artificial neural network trained based on deep learning, or may use an image processing technique. In some embodiments, the artificial neural network trained based on deep learning may be a Locotracker. In some embodiments, the image processing technique may include Lucas-Kanade (LK) optical flow, scale invariant feature transform (SIFT), and speeded-up robust features (SURF). However, an embodiment is not necessarily limited thereto.

The electronic device may calculate coordinates at which vessel points of one frame are located in a next frame by using the motion tracking model. The motion tracking model may calculate a correlation degree of two frames temporally adjacent to each other and may determine coordinates of the vessel points based on the correlation degree. The electronic device may perform point tracking on vessel frames before and after a reference frame based on the reference frame.

Referring to FIG. 7, vessel points PNT_1 to PNT_5 of a first frame FRM_1 among a plurality of image frames may be identified. According to an embodiment, the vessel points PNT_1 to PNT_5 may correspond to points of a centerline CTLN of a reference frame FREF of FIG. 6. That is, the electronic device may determine coordinate data indicating where the points of the centerline CTLN are located in each image frame. In FIG. 7, for convenience of description, reference numerals are indicated only for the vessel points PNT_1 to PNT_5, but the first frame FRM_1 includes other vessel points for which reference numerals are not indicated.

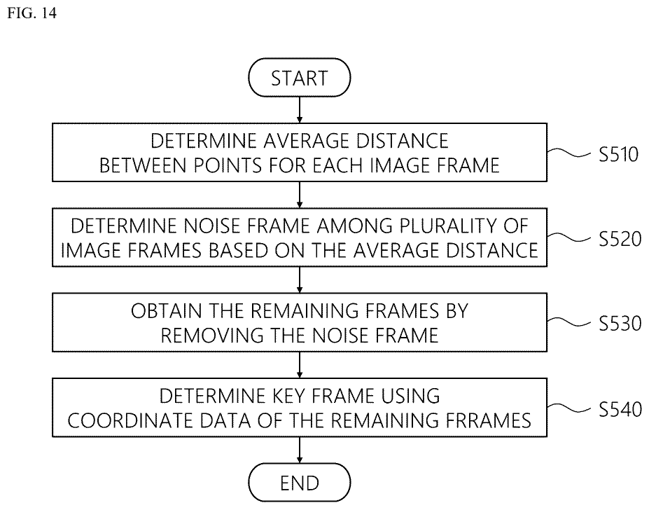

In one embodiment, the electronic device may determine a valid frame based on point tracking in the plurality of image frames. For example, the electronic device may determine the valid frame based on a distance of vessel points. The electronic device may also determine a noise frame based on a distance of vessel points in the plurality of image frames. The electronic device may determine the valid frame by removing the noise frame from the plurality of image frames. A configuration in which the electronic device determines the valid frame will be described later with reference to FIG. 14.

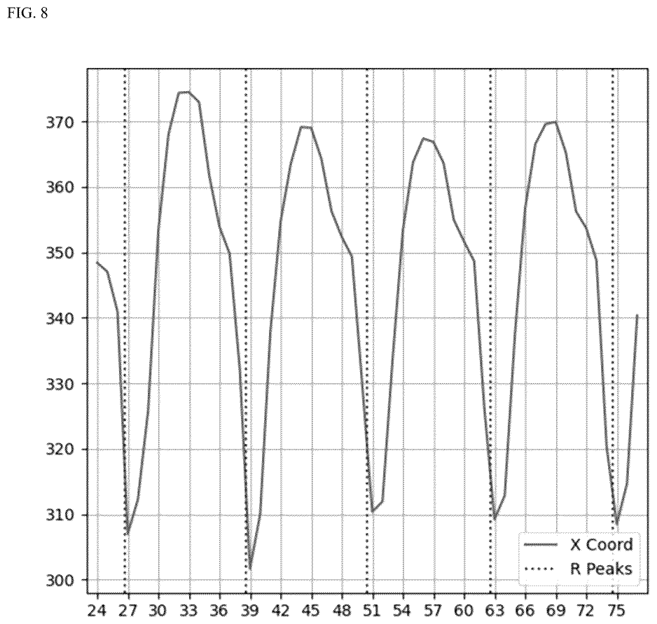

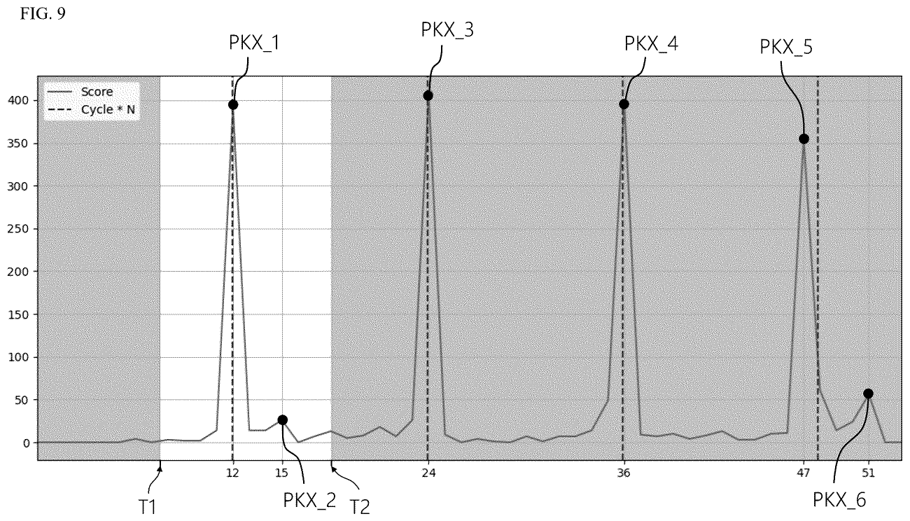

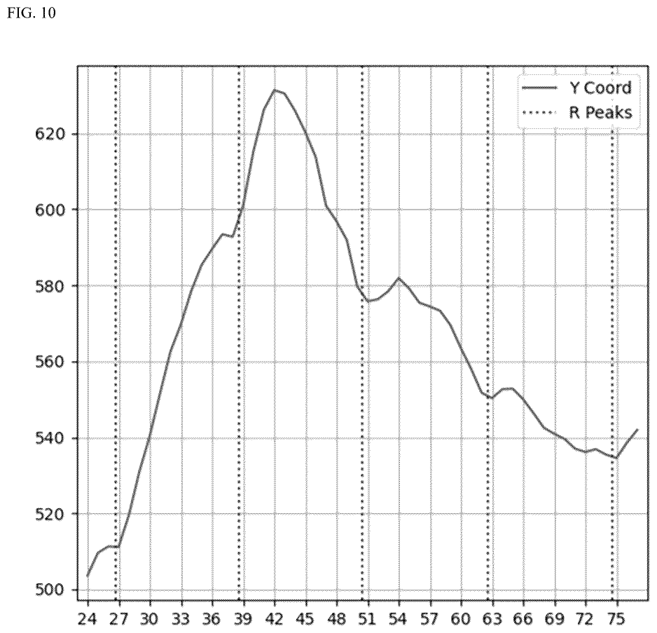

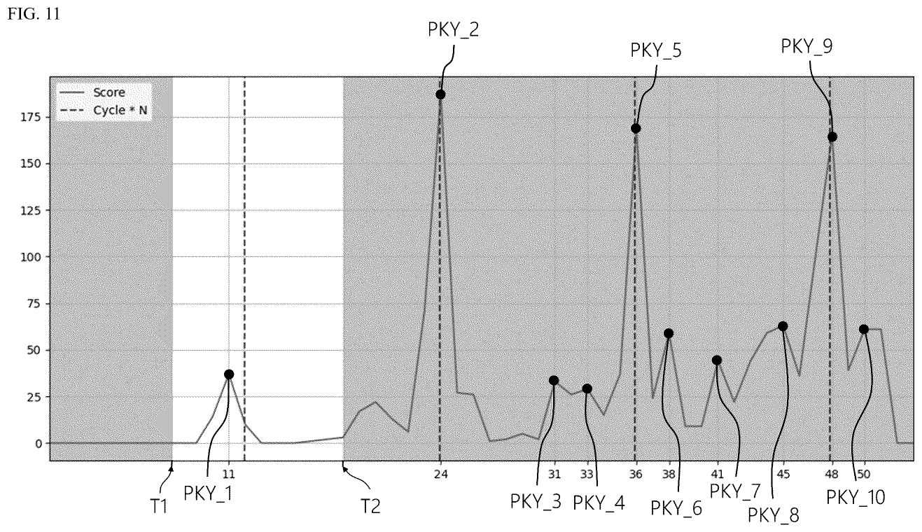

FIG. 8 is a diagram for describing an X-axis coordinate change over time of a vessel point according to one embodiment, FIG. 9 is a diagram for describing a configuration of determining a cardiac cycle from a coordinate change of a vessel point according to one embodiment, FIG. 10 is a diagram for describing a Y-axis coordinate change over time of a vessel point according to one embodiment, and FIG. 11 is a diagram for describing a configuration of determining a cardiac cycle from a coordinate change of a vessel point according to one embodiment.

Referring to FIG. 3, an electronic device may determine a key frame corresponding to a specific cardiac phase based on a time-series variation of coordinate data (S350). For example, the electronic device may determine a cardiac cycle based on the time-series variation of the coordinate data. In one embodiment, the electronic device may use auto-correlation. Auto-correlation is a technique for finding a repeating pattern or periodicity existing between data, and the electronic device may identify a repeating pattern of a plurality of image frames through auto-correlation and may determine the cardiac cycle. However, an embodiment is not necessarily limited thereto, and the electronic device may convert a trajectory of vessel points into a signal and may use an algorithm for detecting periodicity from the signal. For example, the electronic device may also use a Fourier transform, a Wavelet Transform, and Cepstrum Analysis. The electronic device may determine the specific cardiac phase based on the cardiac cycle.

In one embodiment, the electronic device may determine a plurality of candidate periods based on the time-series variation of the coordinate data. The plurality of candidate periods may include periods calculated from each of the vessel points. A period of each vessel point may be an individual candidate period. For example, the electronic device may determine a first period of a first point among the vessel points and may determine a second period of a second point. In this manner, the electronic device may determine periods for the vessel points. The electronic device may determine a dominant period (e.g., a period having a highest frequency) among the plurality of candidate periods as the cardiac cycle.

According to an embodiment, a plurality of periods may also be derived for one point. In this case, the electronic device may determine a period having a highest frequency among all candidate periods obtained from the vessel points as the cardiac cycle.

Referring to FIG. 8, an X-axis coordinate of a first point among vessel points in a plurality of image frames may be identified as changing over time. In the graph, a horizontal axis is time, and a vertical axis represents an X-axis coordinate value of the first point.

In one embodiment, when auto-correlation is used for an X-axis coordinate change of vessel points, it may be as shown in FIG. 9. Referring to FIG. 9, an auto-correlation score of vessel points over time may be identified. In the graph, a horizontal axis is time, and a vertical axis represents the auto-correlation score of the vessel points. The electronic device may detect peaks PKX_1 to PKX_5 that exceed a predetermined score in the graph. The electronic device may determine the cardiac cycle based on the peaks PKX_1 to PKX_5.

Referring to FIG. 10, a Y-axis coordinate of a first point among vessel points in a plurality of image frames may be identified to change over time. In the graph, a horizontal axis is time, and a vertical axis represents a Y-axis coordinate value of the first point.

In one embodiment, when auto-correlation is applied to a Y-axis coordinate change of vessel points, it may be as shown in FIG. 11. Referring to FIG. 11, an auto-correlation score of vessel points over time may be identified. In the graph, a horizontal axis is time, and a vertical axis represents the auto-correlation score of the vessel points. The electronic device may detect peaks PKY_1 to PKY_10 that exceed a predetermined score in the graph. The electronic device may determine the cardiac cycle based on the peaks PKY_1 to PKY_10.

Referring to FIGS. 8 to 11, the electronic device may calculate a period of peaks PKX_1 to PKX_5 and PKY_1 to PKY_10 from an X-axis coordinate change and a Y-axis coordinate change of vessel points. For example, the electronic device may calculate a period between adjacent peaks. The electronic device may determine the most frequently occurring value among calculated periods as the cardiac cycle. For example, the most frequently occurring value refers to a value having a highest frequency. The electronic device may determine a value 12 as the cardiac cycle. The cardiac cycle is indicated as a vertical dashed line in FIGS. 8 to 11.

In embodiments, the electronic device may obtain a normal heart rate of a first user. The normal heart rate may indicate a baseline heart rate or a heart rate in a rest period. The electronic device may determine the cardiac cycle based on the normal heart rate. For example, the electronic device may set a period range based on the normal heart rate. When a value having a highest frequency among calculated periods satisfies the period range, the electronic device may determine the value as the cardiac cycle.

The electronic device may determine a specific cardiac phase based on the cardiac cycle. In one embodiment, the electronic device may obtain first image frames of a time interval greater than the cardiac cycle among a plurality of image frames. For example, as illustrated in FIGS. 9 and 11, the electronic device may obtain the first image frames of a time interval from time point T1 to time point T2. The time interval from time point T1 to time point T2 may be greater than the cardiac cycle. The electronic device may determine a key frame based on coordinate data of vessel points in the first image frames. For example, the electronic device may determine a sinusoidal function indicating a time-series variation of the coordinate data of vessel points in the first image frames. The electronic device may determine the key frame based on the sinusoidal function.

In one embodiment, the electronic device may determine a phase shift in the sinusoidal function by using the cardiac cycle. The electronic device may determine a maximum point or a minimum point of the sinusoidal function based on the phase shift. The electronic device may determine the key frame based on the maximum point or the minimum point. For example, the electronic device may determine the maximum point as the key frame corresponding to a diastolic phase, or may determine the minimum point as the key frame corresponding to a systolic phase.

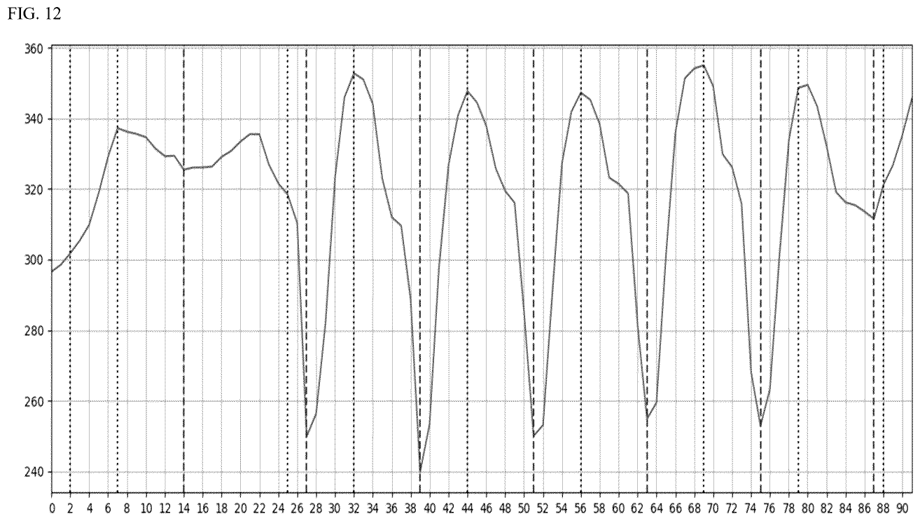

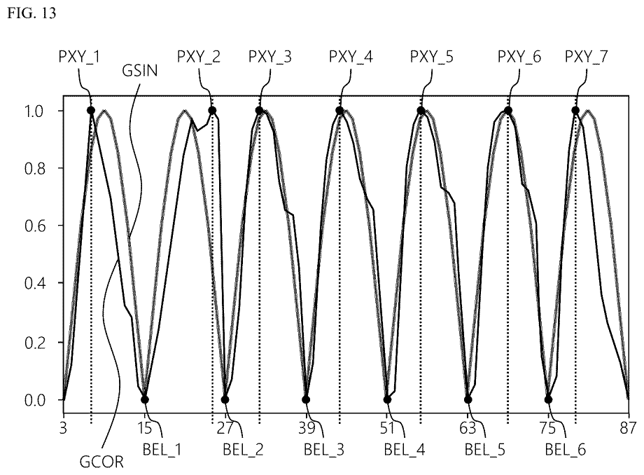

FIG. 12 is a diagram for describing a coordinate change over time of a vessel point according to one embodiment, and FIG. 13 is a diagram for describing a configuration of determining a specific cardiac phase from a coordinate change of a vessel point according to one embodiment.

Referring to FIG. 12, an electronic device according to one embodiment may add an X-axis coordinate change and a Y-axis coordinate change. According to an embodiment, the electronic device may add the X-axis coordinate change and the Y-axis coordinate change by applying different weights to the X-axis coordinate change and the Y-axis coordinate change. In FIG. 12, a horizontal axis is time, and a vertical axis represents a sum of an X-axis coordinate value and a Y-axis coordinate value of a vessel point.

Specifically, the electronic device may obtain an X-axis coordinate and a Y-axis coordinate of vessel points for each image frame through point tracking. The electronic device may calculate an average of the X-axis coordinate and the Y-axis coordinate of the vessel points. In one embodiment, the electronic device may also obtain coordinates of a specific vessel region. For example, the specific vessel region may include at least one of a beginning portion, a middle portion, and a distal portion of a vessel. Since displacement characteristics may be different depending on a vessel region, the electronic device may track vessel points of a region of interest.

The electronic device may generate a time-series graph by repeating calculation of coordinate averages of vessel points in time order. That is, in the graph of FIG. 12, it may be identified that a time point at which a coordinate value reaches a peak and a time point at which the coordinate value reaches a valley repeatedly appear in a specific section, and this may be regarded as reflecting relaxation and contraction phenomena of a vessel according to a cardiac cycle. Through this, the electronic device may obtain a time-series graph that more comprehensively reflects actual displacement characteristics of a vessel. In this case, the time-series graph may exhibit a form of a sinusoidal function.

In one embodiment, the electronic device may determine a section in which peaks and valleys repeatedly appear in the graph of FIG. 12. For example, the electronic device may determine a distance (interval) between adjacent peaks or adjacent valleys, and may determine a section in which a distance between adjacent peaks is within a specific range. The electronic device may generate a time-series graph corresponding to the determined section.

Referring to FIG. 13, a time-series graph GCOR generated by an electronic device according to one embodiment and a sinusoidal function graph GSIN corresponding to the time-series graph GCOR may be identified. The electronic device may generate a sinusoidal function graph GSIN having a variable phase based on a predetermined cardiac cycle. The sinusoidal function graph GSIN may take a form satisfying Equation 1.

$$X ( T ) = A \vert sin ( ω t + ϕ ) \vert + C \qquad [ Equation 1 ]$$

Here, x(t) is an equation of the sinusoidal function graph GSIN, A is an amplitude-related constant for fitting the sinusoidal function graph GSIN to the time-series graph GCOR, C is a minimum-value-related constant for fitting the sinusoidal function graph GSIN to the time-series graph GCOR, ω is an angular frequency corresponding to the cardiac cycle, and φ is a phase shift between the sinusoidal function graph GSIN and the time-series graph GCOR and may be a variable adjusted such that the sinusoidal function graph GSIN and the time-series graph GCOR become as similar as possible. Initially, φ may be a variable phase.

In Equation 1, a sine function is described as an example of the sinusoidal function, but an embodiment is not necessarily limited thereto, and the sinusoidal function may also be implemented as a cosine function or the like.

The electronic device may determine an angular frequency ω based on Equation 2.

$$ω = \frac{2 π}{T} \qquad [ Equation 2 ]$$

Here, ω is an angular frequency corresponding to the cardiac cycle T of the sinusoidal function graph GSIN, and T may be the cardiac cycle.

The electronic device may calculate a correlation coefficient with the time-series graph GCOR for sinusoidal function graphs GSIN having various phase shifts φ. The electronic device may determine a phase shift φ having a greatest correlation coefficient. The electronic device may determine a phase shift φ such that peaks PXY_1 to PXY_7 and valleys BEL_1 to BEL_6 of the time-series graph GCOR correspond to a peak and a valley of the sinusoidal function graph GSIN. Accordingly, the sinusoidal function graph GSIN may be fixed.

The electronic device may determine peaks PXY_1 to PXY_7 and valleys BEL_1 to BEL_6 in the time-series graph GCOR. The electronic device may determine an image frame corresponding to peaks PXY_1 to PXY_7 as a key frame corresponding to diastole. The electronic device may determine an image frame corresponding to valleys BEL_1 to BEL_6 as a key frame corresponding to systole. Accordingly, the electronic device may generate three-dimensional data by using two frames determined as key frames of the same time point.

In one embodiment, the electronic device may also express a degree to which each image frame is close to diastole or systole as a value normalized to a value between 0 and 1. The electronic device may determine a key frame from an image frame based on the normalized value.

In this manner, the electronic device may accurately estimate a cardiac cycle only with a coordinate change inside an image even without a separate sensor. In addition, the electronic device may determine a relatively accurate systolic time point or diastolic time point by flexibly determining an angular frequency ω in an environment in which a heart rate range is estimated to be within a predetermined range. Accordingly, the electronic device may provide information that may be utilized for three-dimensional reconstruction of a vessel or a structure around a heart or subsequent medical care by visually and quantitatively identifying a motion state of a heart.

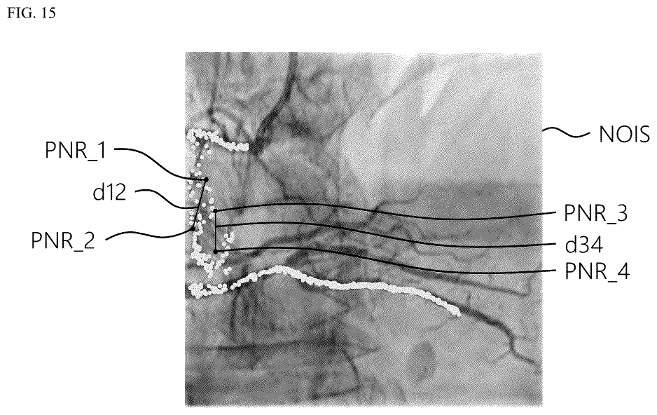

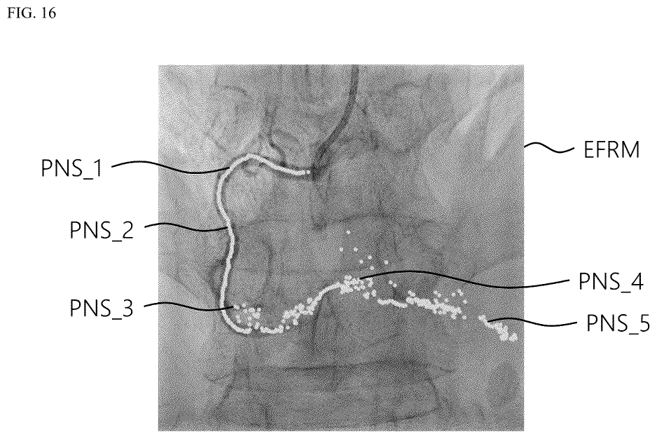

FIG. 14 is a flowchart for describing a noise frame determination method according to one embodiment, FIG. 15 is a diagram for describing a noise frame according to one embodiment, and FIG. 16 is a diagram for describing a valid frame according to one embodiment.

Referring to FIG. 14, a noise frame determination method according to one embodiment may be performed by an electronic device (for example, electronic device 200 of FIGS. 1 and 2). The electronic device may determine an average distance between vessel points for each of a plurality of image frames based on coordinate data (S510). For example, the electronic device may determine vessel points in one image frame based on vascular information. The electronic device may assign an identifier to each vessel point and may track coordinates of the vessel points in the plurality of image frames. In one embodiment, one image frame may be a reference frame. The electronic device may calculate a distance between all point pairs (for example, adjacent points) within the same frame. The electronic device may determine an average distance between vessel points within an image frame by calculating an average of the distances.

The electronic device may determine a noise frame among the plurality of image frames based on the average distance (S520). The electronic device may determine the noise frame based on a predetermined threshold. For example, the electronic device may determine, as the noise frame, a frame in which the average distance between the vessel points is equal to or greater than the threshold among the plurality of image frames.

Referring to FIG. 15, an image frame NOIS among the plurality of image frames may include a plurality of vessel points. The plurality of vessel points may include first to fourth points PNR_1 to PNR_4. The electronic device may determine a distance d12 between the first point PNR_1 and the second point PNR_2 that are adjacent points. The electronic device may determine a distance d34 between the third point PNR_3 and the fourth point PNR_4 that are adjacent points. In this manner, the electronic device may determine distances of adjacent points among the plurality of vessel points and may calculate the average distance from the distances. The electronic device may determine, as the noise frame, the image frame NOIS in which the average distance is equal to or greater than the threshold.

In a normal image frame, the average distance between the vessel points changes within a relatively constant range, but when the distance between points is suddenly calculated to be large only in a specific frame due to shaking during imaging, breathing of a first user, or a point recognition error, the corresponding frame may be determined as a noise frame.

The electronic device may obtain remaining frames obtained by removing the noise frame from the plurality of image frames (S530). That is, among the plurality of image frames, frames having an average distance equal to or greater than (or exceeding) a specific threshold may be regarded as noise frames and excluded, and only the remaining valid frames (or normal frames) may be selected. When a noise frame is included, an abnormal coordinate variation may occur at a specific moment, and an inaccurate cardiac cycle may be determined. Since the electronic device increases consistency of data based on a relatively stable coordinate change by removing the noise frame, accurate analysis may be possible.

Referring to FIG. 16, an image frame EFRM among the plurality of image frames may include a plurality of vessel points. The plurality of vessel points may include first to fifth points PNS_1 to PNS_5. The electronic device may determine distances of adjacent points among the plurality of vessel points and may calculate the average distance from the distances. The electronic device may determine, as a valid frame (or a normal frame), the image frame EFRM in which the average distance is less than the threshold.

The electronic device may determine a key frame by using coordinate data of the remaining frames (S540). The key frame may refer to an image frame representing a specific state corresponding to a diastolic phase or a systole phase. The same description as described above may be applied to a configuration in which the electronic device determines the key frame based on the coordinate data. Accordingly, a redundant description will be omitted. In this manner, the electronic device may identify a key frame with higher reliability by using remaining valid frames in which the noise frame is removed and a normal beating cycle is followed.

In embodiments, steps S510, S520, and S530 may correspond to step S340 of FIG. 3, and step S540 may correspond to step S350 of FIG. 3.

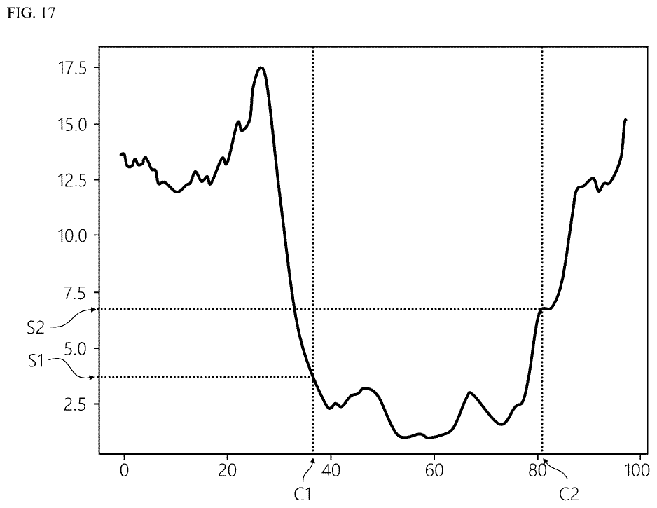

FIG. 17 is a graph for describing a valid frame determination method according to one embodiment.

Referring to FIG. 17, an average distance in each image frame according to one embodiment may be identified. The electronic device may determine a valid frame from the plurality of image frames. The electronic device may determine coordinate data of vessel points for each of the plurality of image frames. The electronic device may determine an average distance between vessel points for each of the plurality of image frames based on the coordinate data. The electronic device may calculate a distance between point pairs (for example, adjacent points) among the vessel points and may calculate an average of the distances. In FIG. 17, a horizontal axis is an image frame number, and a vertical axis may represent the average distance in each image frame.

The electronic device may determine a first time point C1 at which the average distance between the vessel points becomes less than a first threshold S1. The electronic device may determine a second time point C2 at which the average distance between the vessel points becomes equal to or greater than a second threshold S2. According to an embodiment, the first threshold S1 and the second threshold S2 may be set to be the same or different.

The electronic device may select frames from the first time point C1 to the second time point C2 among the plurality of image frames. The electronic device may determine coordinate data in the selected frames. In this manner, the electronic device may identify a key frame with higher reliability by using remaining valid frames that follow a normal beating cycle.

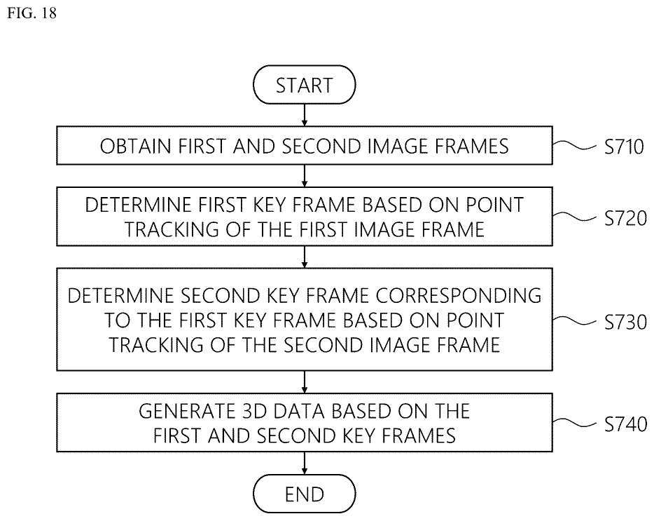

FIG. 18 is a flowchart of a three-dimensional data generation method according to one embodiment.

Referring to FIG. 18, the three-dimensional data generation method according to one embodiment may be performed by an electronic device (for example, electronic device 200 of FIGS. 1 and 2). The electronic device may obtain a plurality of first image frame(s) and a plurality of second image frame(s) captured at different times and different positions (S710). That is, the first image frames may be captured at a first position and a first angle, and the second image frames may be captured at a second position and a second angle. The first position and the second position may be different.

The electronic device may determine a first key frame based on point tracking of the first image frames (S720). For example, the electronic device may determine a first reference frame among the first image frames and may determine vessel points of the first reference frame as first reference points. The electronic device may track position changes of the first reference points in the first image frames.

In one embodiment, the electronic device may determine an average distance between points of the first image frames based on point tracking. The electronic device may determine, as the first key frame, a frame at a point at which the average distance is a maximum or a minimum in the first image frames.

In one embodiment, the electronic device may determine an average distance between points of the first image frames based on point tracking. The electronic device may remove, as a noise frame, a frame in which the average distance is equal to or greater than a threshold in the first image frames. The electronic device may use frames that remain without being removed in the first image frames as valid frames (or normal frames). The electronic device may determine the first key frame in the valid frames.

In one embodiment, the electronic device may determine a cardiac cycle based on point tracking. The electronic device may determine the first key frame based on the cardiac cycle. The electronic device may generate a time-series graph indicating repetitiveness based on the cardiac cycle and may determine, as the first key frame, a frame corresponding to a peak or a valley of the time-series graph.

The electronic device may determine a second key frame corresponding to the first key frame based on point tracking of the second image frames (S730). For example, the electronic device may determine a second reference frame among the second image frames and may determine vessel points of the second reference frame as second reference points. The electronic device may track position changes of the second reference points in the second image frames. The electronic device may determine the second key frame similarly to a configuration of determining the first key frame.

In one embodiment, when the first key frame is a frame of a diastolic phase at a first time point, the electronic device may determine the second key frame corresponding to the diastolic phase at the first time point in the second image frames. When the first key frame is a frame of a systolic phase at a second time point, the electronic device may determine the second key frame corresponding to the systolic phase at the second time point in the second image frames.

The electronic device may generate three-dimensional data based on the first key frame and the second key frame (S740). The electronic device may generate an FFR value with the three-dimensional data. In this manner, the electronic device may rapidly and precisely generate the three-dimensional data even with reduced data and computational resources by determining two key frames corresponding to a specific cardiac phase from images without additional data.

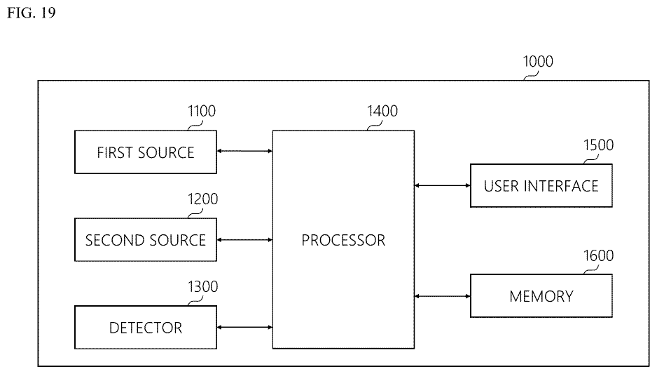

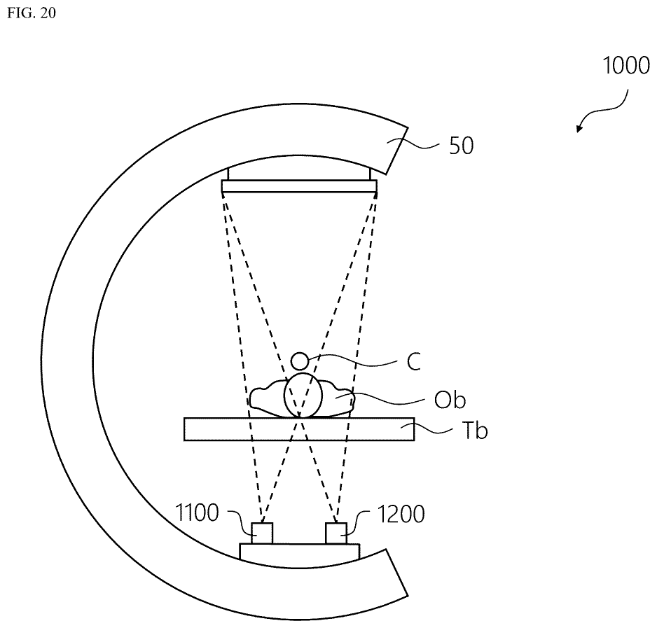

FIG. 19 is a block diagram of an imaging system according to one embodiment, and FIG. 20 is a diagram for describing a structure of the imaging system according to one embodiment.

Referring to FIGS. 19 and 20, an imaging system 1000 according to one embodiment may capture an object Ob placed (arranged) on a table Tb to generate a stereo image. The stereo image may be an image including a blood vessel of the object Ob and may be used to generate three-dimensional vascular data.

The imaging system 1000 includes a first source 1100 configured to irradiate X-rays, a second source 1200 configured to irradiate X-rays at a different position and a different angle from the first source 1100, a detector 1300 configured to detect X-rays that are irradiated from each of the first source 1100 and the second source 1200 and pass through the object Ob, at least one processor 1400 configured to determine an image based on the detected X-rays, a user interface 1500 configured to receive a command from a user or output the image, and a memory 1600 configured to store various information and images required for control.

Hereinafter, an example will be described in which the imaging system 1000 is provided as a C-arm X-ray image apparatus including a C-arm 50 as shown in FIG. 20 and is used for angiography.

However, the type and utilization of the imaging system 1000 are not limited to the following description, and the imaging system 1000 may be implemented as various types of imaging systems and used for various clinical applications. For example, in some embodiments, the imaging system 1000 may be implemented as a mono-plane device or a bi-plane device.

As shown in FIG. 20, the first source 1100 and the second source 1200 may be provided at one end of the C-arm 50, and the detector 1300 may be provided at the other end of the C-arm 50 to face the first source 1100 and the second source 1200.

In this case, the first source 1100 and the second source 1200 may be X-ray sources of a known type, and each of the first source 1100 and the second source 1200 may irradiate separate X-rays to the object Ob so as to capture an X-ray image of the object Ob at different positions and different angles. According to an embodiment, the imaging system 1000 according to one embodiment may include one movable source, and the source may irradiate X-rays at various positions and angles while moving on the C-arm 50.

However, an embodiment is not necessarily limited thereto, and the first and second sources 1100 and 1200 may be implemented in various manners depending on a type of an imaging device, such as ultrasound, radio waves, infrared rays, etc., rather than X-rays.

The detector 1300 is a component for detecting X-rays that are irradiated from the sources 1100 and 1200 and pass through the object Ob and may be an X-ray detector of a known type. For example, the detector 1300 may include a scintillator (not shown), a photodiode (not shown), and a storage element (not shown), but an embodiment is not necessarily limited thereto.

The at least one processor 1400 according to one embodiment may control an overall operation of the imaging system 1000. For example, the processor 1400 may determine an image based on X-rays detected through the detector 1300.

The at least one processor 1400 according to one embodiment may determine a cardiac cycle based on a first image corresponding to the first source 1100 and a second image corresponding to the second source 1200. The at least one processor 1400 may determine key frames corresponding to a specific cardiac phase from each of the first image and the second image based on the cardiac cycle.

The at least one processor 1400 may generate three-dimensional data based on the key frames. The at least one processor 1400 may use location information of each of the first source 1100, the second source 1200, and the detector 1300, and imaging parameters of each of the first source 1100 and the second source 1200. In this case, the location information may be information about relative locations of the respective components. For example, the location information may include a first location at which the first source 1100 irradiates first X-rays, a second location at which the second source 1200 irradiates second X-rays, and third and fourth locations at which the detector 1300 detects the first X-rays and the second X-rays, respectively.

The imaging parameters may include a capturing position of the sources 1100 and 1200, a capturing angle, a capturing distance, a position and an angle of the table Tb, a focal length, a baseline distance, a capturing direction (axial rotation information), a pixel spacing, relative position and direction information between the sources 1100 and 1200, a center point C of the C-arm 50, and the like. The at least one processor 1400 may calculate an FFR value of a blood vessel from the three-dimensional data.

A user interface 1500 according to one embodiment may include an input device configured to receive a user input and a display configured to output an image, and according to an embodiment, the input device and the display may be implemented as a touch screen module provided integrally. For example, the user interface 1500 may receive a command for capturing an image and may receive an input of selecting a feature point in an image.

Further, the user interface 1500 may display each of a first image obtained by the first source 1100 and a second image obtained by the second source 1200, may display three-dimensional data in which the first image and the second image corresponding to two-dimensional images are combined, and may display an FFR value calculated from the three-dimensional data.

A memory 1600 according to one embodiment may be provided as a memory of a known type so as to store various information and images required for an operation of the processor 1400.

In some embodiments, each component or a combination of two or more components described with reference to FIG. 1 to FIG. 20 may be implemented with digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, LED (light-emitting diode) monitor, OLED (organic LED) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. An electronic device, comprising:
a processor; and
a memory connected to the processor,
wherein the memory is configured to store a program,
wherein the processor is configured to execute the program, and
wherein, when the program is executed, the electronic device is configured to:
obtain a plurality of image frames;
determine one of the plurality of image frames as a reference frame;
determine vessel points in the reference frame;
for each of the plurality of image frames, determine coordinate data of the vessel points; and
determine a key frame corresponding to a specific cardiac phase based on a time-series variation of the coordinate data.

2. The electronic device of claim 1, wherein determining one of the plurality of image frames as the reference frame comprises:
determining the reference frame based on a contrast agent distribution level and a vessel position score in the plurality of image frames.

3. The electronic device of claim 1, wherein determining the vessel points in the reference frame comprises:
segmenting a vessel region in the reference frame;
determining a skeleton in the vessel region; and
determining the vessel points based on the skeleton.

4. The electronic device of claim 1, wherein determining the coordinate data of the vessel points for each of the plurality of image frames comprises:
determining the coordinate data by tracking a positional change of the vessel points in the plurality of image frames using a motion tracking model.

5. The electronic device of claim 1, wherein determining the coordinate data of the vessel points for each of the plurality of image frames comprises:
for each of the plurality of image frames, determining an average distance between the vessel points based on the coordinate data;
determining a noise frame among the plurality of image frames based on the average distance; and
obtaining remaining frames obtained by removing the noise frame from the plurality of image frames, and
wherein determining the key frame corresponding to the specific cardiac phase based on the time-series variation of the coordinate data comprises:
determining the key frame using coordinate data of the remaining frames.

6. The electronic device of claim 5, wherein determining the noise frame among the plurality of image frames based on the average distance comprises:
determining, as the noise frame, a frame among the plurality of image frames in which the average distance between the vessel points is greater than or equal to a threshold.

7. The electronic device of claim 1, wherein determining the coordinate data of the vessel points for each of the plurality of image frames comprises:
for each of the plurality of image frames, determining an average distance between the vessel points based on the coordinate data;
determining a first time point at which the average distance between the vessel points becomes less than a threshold;
determining a second time point at which the average distance between the vessel points becomes greater than or equal to the threshold;
selecting, from the plurality of image frames, frames from the first time point to the second time point; and
determining the coordinate data in the selected frames.

8. The electronic device of claim 1, wherein determining the key frame corresponding to the specific cardiac phase based on the time-series variation of the coordinate data comprises:
determining a cardiac cycle based on the time-series variation of the coordinate data; and
determining a diastolic phase or a systolic phase based on the cardiac cycle.

9. The electronic device of claim 8, wherein determining the cardiac cycle based on the time-series variation of the coordinate data comprises:
determining a plurality of candidate periods based on the time-series variation of the coordinate data; and
determining, as the cardiac cycle, a period having a highest frequency among the plurality of candidate periods.

10. The electronic device of claim 8, wherein determining the diastolic phase or the systolic phase based on the cardiac cycle comprises:
obtaining first image frames within a time interval that is greater than the cardiac cycle among the plurality of image frames; and
determining the key frame in the first image frames based on coordinate data of the vessel points in the first image frames.

11. The electronic device of claim 10, wherein determining the key frame in the first image frames based on the coordinate data of the vessel points comprises:
determining, in the first image frames, a sinusoidal function representing a time-series variation of the coordinate data of the vessel points; and
determining the key frame based on the sinusoidal function.

12. The electronic device of claim 11, wherein determining the key frame based on the sinusoidal function comprises:
determining a phase shift in the sinusoidal function using the cardiac cycle;
determining a maximum point or a minimum point of the sinusoidal function based on the phase shift; and
determining the key frame based on the maximum point or the minimum point.

13. The electronic device of claim 12, wherein determining the key frame based on the maximum point or the minimum point comprises:
determining the maximum point as the key frame corresponding to the diastolic phase; or
determining the minimum point as the key frame corresponding to the systolic phase.

14. The electronic device of claim 1, further comprising generating 3D data using two frames determined as key frames at the same time point.

15. An electronic device, comprising:
a processor; and
a memory connected to the processor,
wherein the memory is configured to store a program,
wherein the processor is configured to execute the program, and
wherein, when the program is executed, the electronic device is configured to:
obtain a first image frame and a second image frame captured at different times and different positions;
determine a first key frame based on point tracking of the first image frame;
determine a second key frame corresponding to the first key frame based on point tracking of the second image frame; and
generate 3D data based on the first key frame and the second key frame.

16. The electronic device of claim 15, wherein determining the first key frame based on the point tracking of the first image frame comprises:
determining an average distance between points in the first image frame based on the point tracking; and
determining, as the first key frame, a frame at a point at which the average distance is at a maximum or a minimum in the first image frame.

17. The electronic device of claim 15, wherein determining the first key frame based on the point tracking of the first image frame comprises:
determining an average distance between points in the first image frame based on the point tracking; and
removing, as a noise frame, a frame in which the average distance is greater than or equal to a threshold in the first image frame.

18. The electronic device of claim 15, wherein determining the first key frame based on the point tracking of the first image frame comprises:
determining a cardiac cycle based on the point tracking; and
determining the first key frame based on the cardiac cycle.

19. The electronic device of claim 15, wherein determining the second key frame corresponding to the first key frame based on the point tracking of the second image frame comprises:
when the first key frame is a frame of a diastolic phase at a first time point, determining, in the second image frame, the second key frame corresponding to the diastolic phase at the first time point; and
when the first key frame is a frame of a systolic phase at a second time point, determining, in the second image frame, the second key frame corresponding to the systolic phase at the second time point.

20. A frame determination method, comprising:
obtaining a plurality of image frames;
determining one of the plurality of image frames as a reference frame;
determining vessel points in the reference frame;
for each of the plurality of image frames, determining coordinate data of the vessel points; and
determining a key frame corresponding to a specific cardiac phase based on a time-series variation of the coordinate data.
